(19) United States
(12) Patent Application Publication
(10) Pub. No.: US 2018/0153658 A1
(43) Pub. Date: Jun. 7, 2018

(54) ANATOMICAL ARTICULATOR FOR DENTAL DIAGONOSTIC METHOD AND PROSTHETIC RECONSTRUCTION

(71) Applicant: Ohio State Innovation Foundation, Columbus, OH (US)

(72) Inventor: Shereen S. Azer, Lewis Center, OH (US)

(21) Appl. No.: 15/832,298

(22) Filed: Dec. 5, 2017

Related U.S. Application Data
(60) Provisional application No. 62/430,058, filed on Dec. 5, 2016.

(51) Int. Cl. A61C 11/02; A61C 19/045; A61B 6/03; A61B 6/00

(52) U.S. Cl. CPC A61C 11/02; A61B 6/4085; A61B 6/032; A61C 19/045

(57) ABSTRACT

An anatomical articulator for modeling a temporomandibular joint includes a lower base portion comprising at least one upright support. A set of replica condyles produced from digital data representing a patient jaw structure is connected to the at least one upright support. An upper frame portion defines receptacles on either side. A set of replica mandibular fossae produced from the digital data representing a patient jaw structure is configured to connect to the upper frame portion via the receptacles. The replica condyles and the replica mandibular fossae meet to form a hinge of the articulator.

### CROSS REFERENCE TO RELATED APPLICATIONS

This application claims priority to and incorporates entirely by reference U.S. Provisional Patent Application Ser. No. 62/430,058 filed on Dec. 5, 2016, and entitled “Anatomical Articulator for Dental Diagnostic Methods and Prosthetic Reconstruction.”

### STATEMENT REGARDING FEDERALLY SPONSORED RESEARCH OR DEVELOPMENT

No federally sponsored research or development has been used for this disclosure.

### FIELD OF THE INVENTION

This disclosure relates to the field of apparatuses for modeling the temporomandibular joint of a living being, particularly humans, and diagnosing dental procedures to alleviate disorders of the temporomandibular joint, as well as for prosthetic reconstruction of the masticatory system.

### BACKGROUND

The temporomandibular joint is a bone structure in the jaw that provides a hinge that connects the jaw to the skull. This joint is the structure by which a jaw moves up and down and side to side for chewing, speaking, and facilitating facial movements. The joint is characterized by condyles of the lower jaw fitting within the mandibular fossae of the temporal bone of a patient's head. Problems with the jaw and the muscles in one's face that control it are known as temporomandibular disorders (TMD).

Dentists, surgeons, and other diagnosticians model the mandible (lower jaw) and the upper jaw (or maxilla) in relation to the temporomandibular joint (TMJ) to address the way that this overall assembly moves. Upon determining the way that a patient's jaw structure moves, physicians and dentists can effectively plan surgical or dental relief for a temporomandibular disorder (TMD).

As a simple summary, the medical team treating a patient first makes a model of portions of an upper and lower jaw accessible from the mouth, including the basic gum and teeth structures in a patient's mouth. This model is often created from dental molds taken from a patient's bite structure, and more often in modern clinics, the model may be created from computerized tomography (CT) scans of the inside portion of a patient's mouth and jaw. These models of the patient's mouth (i.e., portions of the mandible and maxilla inside the mouth) have traditionally been created from inexpensive molds of plastics, elastomeric polymers, or plasters taken while the patient bites down on an impression assembly. The mold is used to create a cast that models the patient's bite and mouth. Modeling the bone structure of the jaw, of course, has traditionally been much more difficult, given that no internal impression is available for making a mold and associated cast of an individual's internal skull, bones, and joints. Accordingly, the apparatuses used to approximate the joint movement corresponding to a given dental cast have been entirely generic and not individualized for a patient's actual jaw as it exists in real life.

It is important to recall, as noted above, that one goal of the efforts in this area of medicine is to model and emulate a patient's bite and mouth structure as it moves pursuant to a corresponding TMJ structure. Prior efforts in this regard include building the model of the patient's bite and attaching the upper and lower bite casts to appropriate portions of an apparatus such that the assembly emulates the movement of a patient's jaw. The assembled apparatus has been commonly referred to as an articulator, and the articulator is a hinged device with an upper portion (or upper member) connected to the modeled cast of the upper jaw and a lower portion (lower member) connected to the model of the lower jaw. By moving the upper jaw and lower jaw casts with the hinged articulator, the diagnostician can approximate the patient's modeled mouth and teeth positions for planning corrective action.

Several views of the Figures herein illustrate prior art articulators that have been used for modeling jaw movements for various patient casts. FIG. 1 shows a prior art version of a dental articulator in which a lower base (109) connects to an upper frame (105). The base (109) and frame (105) are hinged to allow rotational movement about an axis running along a horizontal connector (105A) that connects to a front extension (105B) of the upper frame (105). An underside of the front extension (105B) is typically used as a mount for the upper jaw cast. The upper portion has dials (115) on either end of the horizontal connector (105A). The dials allow the diagnostician to adjust the dimensions in an x, y, and z plane to match measurements of a patient's jaw structure prior to attaching the dental casts to the articulator.

FIG. 2 is a prior art image of a lower base of a standard articulator. FIG. 2 shows a side perspective view of the lower base (209) with lower base body (209C) supporting opposite uprights (209A, 209B) and a horizontal connector (209D) extending there between. An elevated platform (211) extends from the lower base (209) and along with a fixation material (207) is used to mount a cast of the lower jaw (not shown). FIG. 2 is characterized by showing mechanical replicas of condyles (202A, 202B) of a patient's mandible. The replicas include mounts extending from the horizontal connector (209D) toward respective spherical termini. The spherical termini approximate the upper end of a patient's condyles for meeting mechanical replicas of a patient's mandibular fossae on either side of the jaw. Prior art FIG. 6 illustrates the mechanical replica of the mandibular fossae (606A) into which a mechanical replica of the condyles with spherical terminus (602A) fits. As illustrated in FIG. 6, the mechanical replicas of the mandibular fossae (606), are portions of the upper frame (105/605) and are predominantly sharply angled imitations of mandibular fossae meeting the spherical termini (602) of the mechanical replicas of the mandible condyles.

In the prior art embodiments of the articulators known to date, the connection of the replicated condyles (FIG. 2, Ref. 202) and the replicated mandibular fossae (FIG. 6, Ref. 606) are allowed three degrees of freedom in the x, y, z directions. The diagnostician utilizes a control rod (113, 213, 313) to move the upper frame (105, 305) relative to the lower base (309). By managing the measurement adjustments available from dials (115) and the position of a control rod (113, 313), the articulator carrying a mounted upper and lower jaw cast pair can provide a general attempt to emulate the TMJ and associated reaction of a patient's bite. The biggest limitation of this set up lies in the generic temporomandibular joint (TMJ) replicas shown in FIG. 6. A pair of simple spheres, as used for the replicas of condyles (202A, 202B), is ineffective to show the exact movement of a true condyle, and that issue is compounded by the crude nature of the mandibular fossae replica (606A) that includes none of the bone structures that would affect the naturally occurring TMJ and a true condyle-fossae relationship in a live patient. Prior art FIGS. 3-5 show more elaborate examples of the prior art articulators described above with casts (314) mounted therein.

A need in the art of dental articulators includes a more anatomically accurate replica of the TMJ so that articulators can be utilized with less cumbersome measurements and dials and more accurate depictions of a patient's temporomandibular disorder.

### BRIEF SUMMARY OF THE INVENTION

An anatomical articulator for modeling a temporomandibular joint (TMJ) includes a lower base portion comprising at least one upright support. A set of replica condyles produced from digital data representing a patient jaw structure is connected to the at least one upright support. An upper frame portion defines receptacles on either side. A set of replica mandibular fossae produced from the digital data representing a patient jaw structure is configured to connect to the upper frame portion via the receptacles.
The replica condyles and the replica mandibular fossae meet to form a hinge of the articulator.

### BRIEF DESCRIPTION OF SEVERAL VIEWS OF THE DRAWINGS

FIG. 1 is a prior art articulator of previously known construction.

FIG. 2 is a lower base of a prior art articulator similar to that of FIG. 1.

FIG. 3 is a prior art articulator of previously known construction having a dental cast mounted therein.

FIG. 4 is a prior art articulator of previously known construction illustrating movement of a temporomandibular joint replica hinging the articulator and having a dental cast mounted therein.

FIG. 5A is a rear view of the prior art articulator of FIG. 4.

FIG. 5B is a another rear view of the prior art articulator of FIG. 4 showing the spherical condyle termini of the replica and the angled fossae replicas of the temporomandibular joint.

FIG. 6 is a close up side view of a prior art spherical condyle termini of the replica and the angled fossae replicas of the temporomandibular joint.

FIG. 7 is a side view of an articulator according to the description herein having printed portions of an upper frame of the articulator with printed condyles and mandibular fossae incorporated therein.

FIG. 8 is a prior art articulator of previously known construction having a maxillary (upper) dental cast mounted therein.

FIG. 9 is a side perspective view of an articulator according to the description herein having printed portions of an upper frame of the articulator with printed condyles and mandibular fossae incorporated therein along with a front extension and control rod portions of an upper frame.

FIG. 10 is a front perspective view of a lower base portion of a prior art articulator showing the spherical condyle termini of the TMJ replica and an elevated platform for mounting a lower jaw cast thereon.

FIG. 11 is a front perspective view of an articulator according to this description incorporating a lower base portion with a mounting platform for a lower jaw cast and printed components including a patient's replicated condyles, mandibular fossa, and a horizontal connector extending between the condyles.

FIG. 12 is a prior art lower base of a prior art articulator.

FIG. 13A is a prior art model of a human jaw illustrating a typical formation of a patient's condyles.

FIG. 13B is a front plan view of an articulator according to the description herein having printed portions of lower base of the articulator with printed condyles extending from a horizontal connector attached to uprights forming part of a lower base structure for the articulator.

FIG. 14 is a side perspective view of prior art articulator according to the description herein.

FIG. 15 is a side perspective view of an articulator according to the description herein having printed portions of an upper frame of the articulator with printed condyles and mandibular fossae incorporated therein.

FIG. 16 is a bottom perspective view of the articulator of FIG. 11.

FIG. 17 is a side perspective view of the articulator of FIG. 11.

FIG. 18 is a side view of the articulator of FIG. 11.

FIG. 19 is a bottom perspective view of the articulator of FIG. 11.

FIG. 20 is a bottom view of the articulator of FIG. 11.

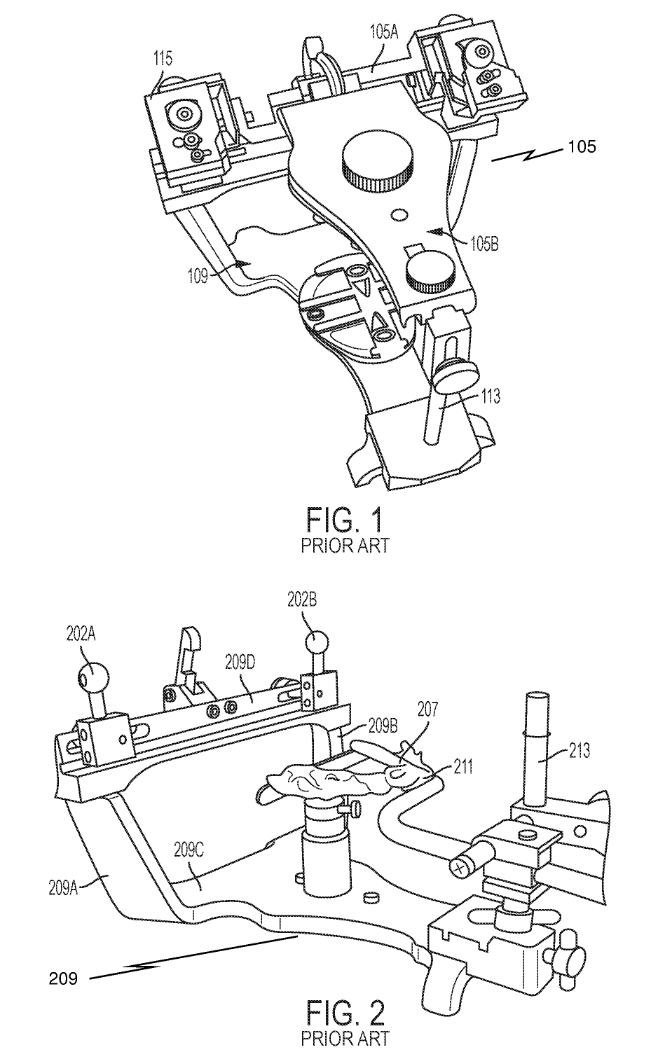

### DETAILED DESCRIPTION

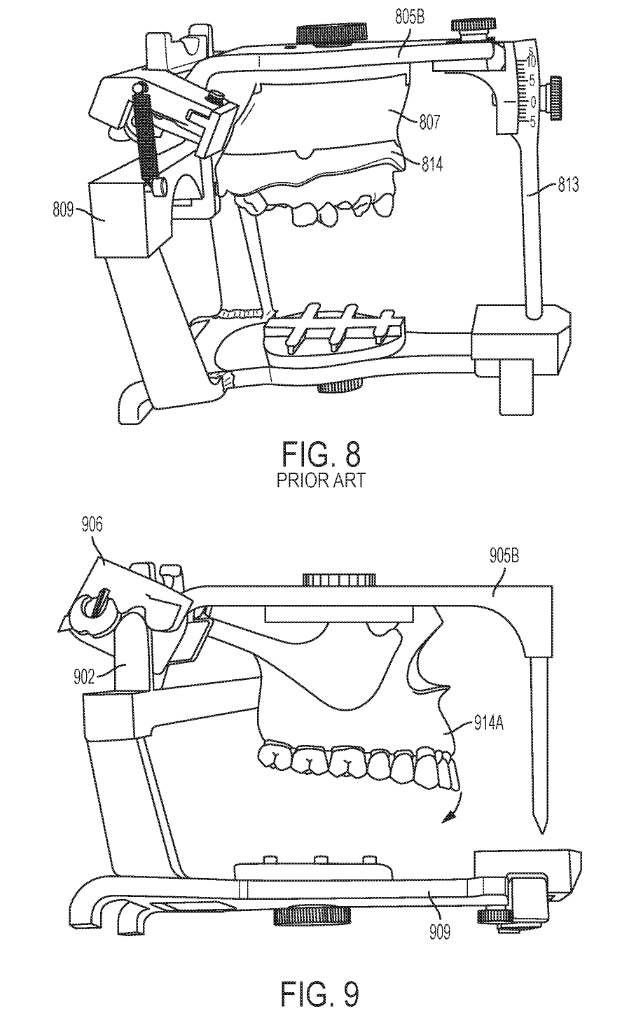

In one embodiment, an articulator, according to the disclosure herein, includes common portions of standard articulators with significant changes in the replicated temporomandibular joint (TMJ). Instead of the above noted prior art replicas with generic and ineffective condyle termini in the form of simple spheres (202) and right angled or at least sharply angled fossae (606), one embodiment of an articular (900) includes a lower base (909) on which a lower jaw (not shown) would be mounted on the above-noted elevated platform. Instead of the mechanical replicas described above, however, the articulator of FIG. 9 includes an exact replica of a patient's condyles (902) and fossae (906) that have been printed via stereolithographic three-dimensional printing techniques. Accordingly references to anatomically modeled or printed condyles and fossae may be considered “replica condyles” and “replica fossae.” Other additive layer three-dimensional printing options are also available to create accurate models of the TMJ condyles and fossae from digital data acquired from scanned images of a patient's bone structure. In one manufacturing method associated with this apparatus, a medical practitioner or diagnostician obtains a cone beam computed tomography (CBCT) scan of the patient's jaw, temporomandibular joint (TMJ), bite structure, and teeth, resulting in highly accurate digital scans and related digital images of the patient's teeth, jaw, and joint structure. These scans can be electronically manipulated to isolate portions of the patient's skull, temporal bone, fossae, and condyles for the most accurate models to be formed from this data.

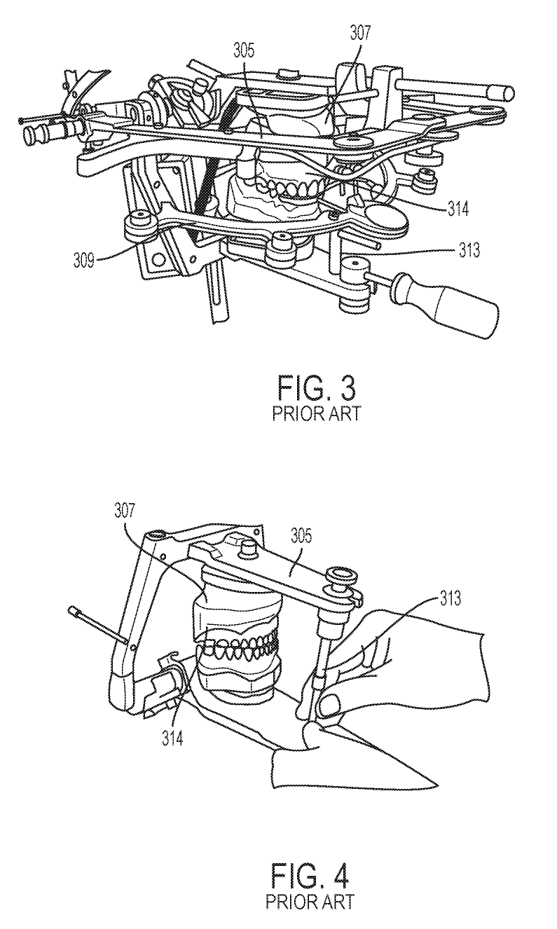

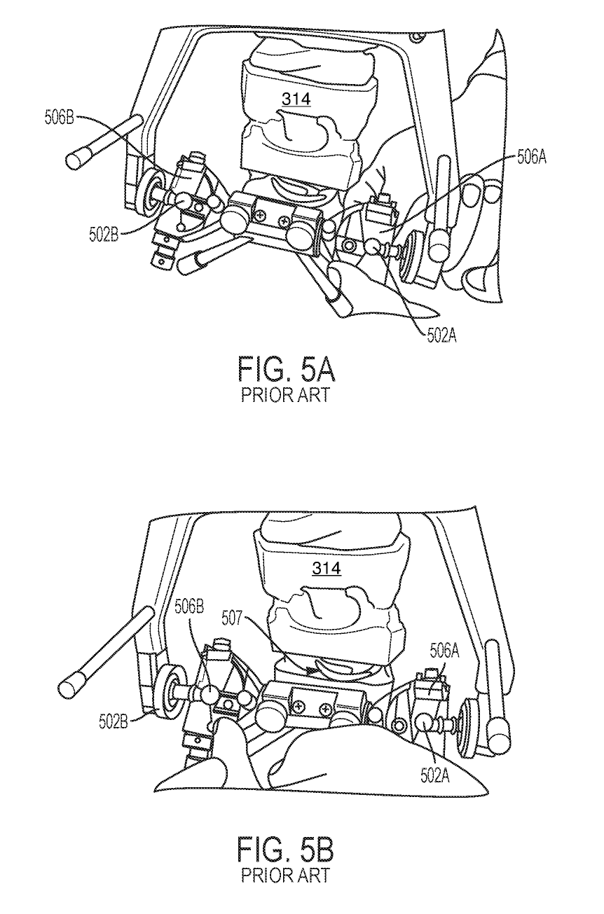

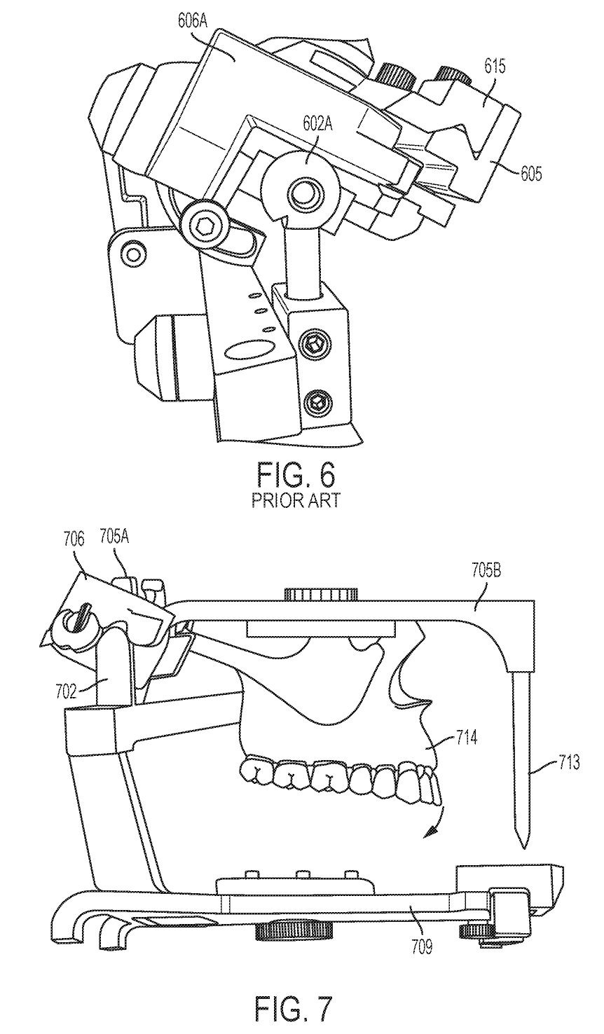

As shown in FIGS. 7 and 9, in one aspect, an articulator may incorporate the printed models of the condyles (702, 902) and the fossae (706, 906) into standard articulator portions including the lower base (709, 909) and upper frame (705, 905). The condyles (702, 902) and the fossae (706, 906) are exact, printed replicas of a patient's CBCT scan. The structures are complete in that they model the TMJ exactly, as opposed to prior art spheres and angles discussed above in regard to condyle and fossae models. In the embodiments of FIGS. 7 and 9, the horizontal connector (709D, 909D) is also printed to connect the printed condyles (702, 902). The horizontal connector (709D, 909D) is configured to connect to uprights (709A, 709B, 909A, 909B). In this embodiment, a patient's 3D print of condyles (702, 902) and horizontal connector (709D, 909D) extending between the condyles are temporarily attachable and completely removable from the lower base uprights. The condyle and connector assembly is modular and most likely disposable after use for a patient. The lower base portion is re-used after cleaning for the next mounted lower jaw (similar to those of FIGS. 3-5) and individualized condyles as shown in FIG. 7 and FIG. 9.

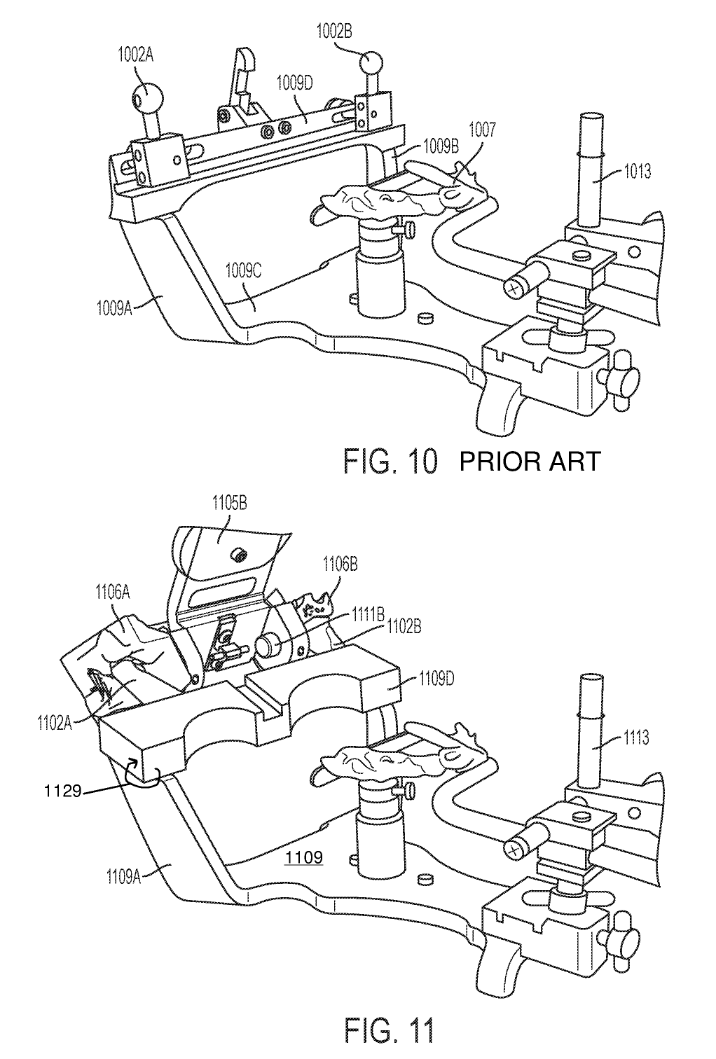

FIGS. 8, 10, 12, 13A, and 14 present prior art embodiments that are useful in side-by-side comparisons of FIGS. 9, 11, 13B, 12, and 15. Corresponding parts in each of these prior art figures have reference numbers corresponding to the appropriate page of drawings such that the drawings show previous versions of condyles (1002A, 1002B, 1202A, 1202B, 1402A, 1402B), fossae, lower base of an articulator (809, 1009, 1209, 1409), upper frame of an articulator (805B), cast structures of dentistry (807, 814), as well as fixing material (1007, 1207), guiding rod (813. 1013, 1213. 1413), horizontal connectors (1009D, 1209D, 1409D) and uprights (1009A, 1009B, 1209A, 1209B. 1409A, 1409B) for a respective articulator assembly.

FIG. 11 shows more details of the embodiment of the lower base (1109) described above for FIGS. 7 and 9. As shown in the image of FIG. 11, the printed condyles (1102A, 1102B) and horizontal connector (1109D) fit onto a lower base (1109) that includes a platform for mounting a lower jaw cast, and a control rod (1113) allows a diagnostician to manipulate an overall model of a patient's jaw structure from an attached upper frame (1105). The upper frame (1105) is configured to attach to 3D printed fossae (1106) via a rotatable axles (1111) that fit within corresponding receptacles of the upper frame (1105). FIG. 11 illustrates one design of the horizontal connector (1109D) may also be rotatable about uprights (1109A, 1109B) to yield another degree of freedom for parts of the articulator to be adjusted for any given patient (e.g., see the arrow (1129) noting that the horizontal axis can be rotated to bring the upper frame, condyles and fossae into a vertical position relative to a horizontal orientation of the base (1109). In other words, regardless of the shape or orientation of a design for the horizontal connector (1109D), the condyles (1102A, 1102B) can be positioned to vertical orientations when the base (1109) is considered a horizontal. The condyles (1102A, 1102B) move just as allowed in the human patient jaw because the associated fossae (1106A, 1106B) have also been printed to match the patient's TMJ structure in the patient's real anatomy. As can be seen in FIG. 13A, a real anatomy for a TMJ structure is not perfectly spherical such as condyles of prior art implementations and/or not perfectly angled such as fossae of the prior art implementations.

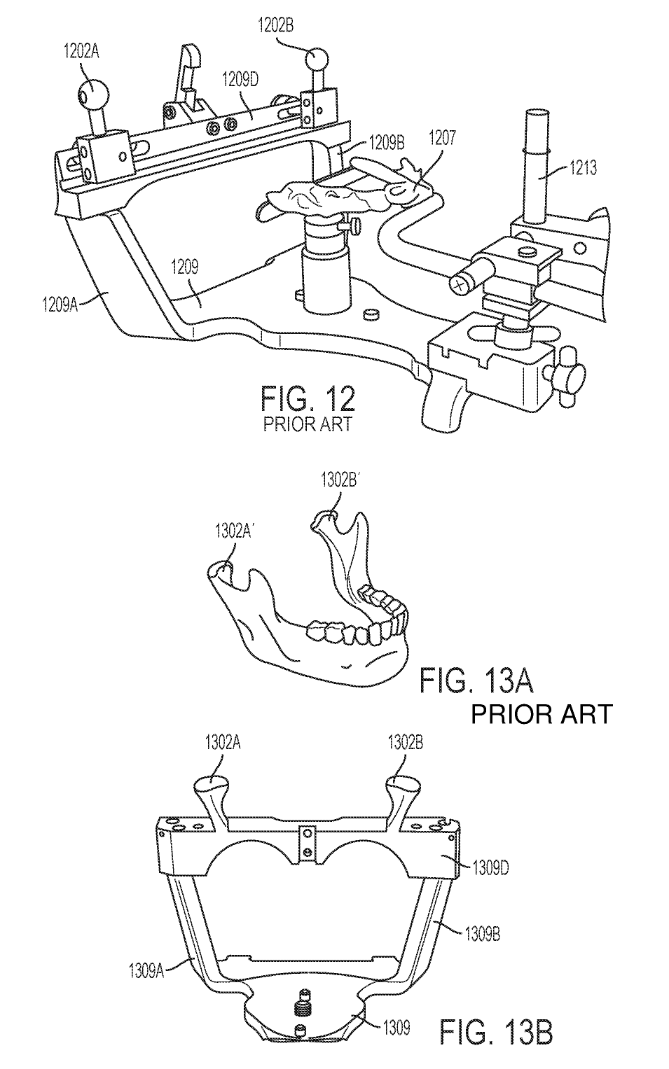

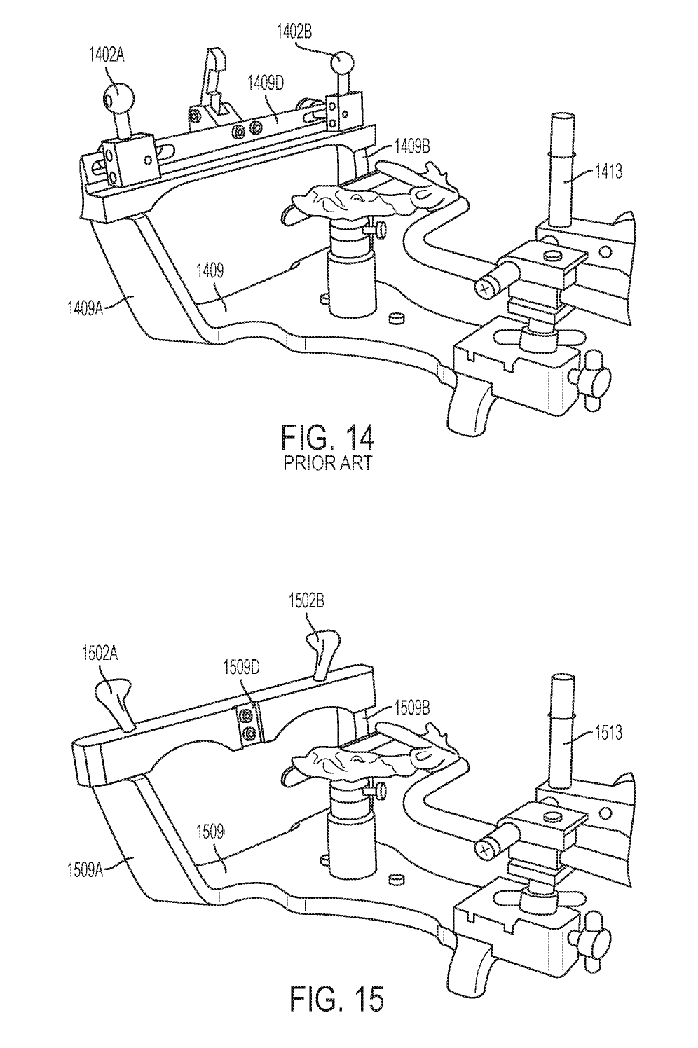

FIG. 13B illustrate printed 3D articulator components as discussed above in relation to a lower base portion (1309) of a prior art articulator of FIG. 12 and an exact replica of a lower jaw from a human anatomy as shown in FIG. 13A. In FIG. 13B, the articulator according to this description incorporates both permanent, reusable portions of a lower base of an articulator with uprights (1309A, 1309B) receiving a printed version of a patient's actual condyle structures (1302) connected by a horizontal connector that is also printed, forming a single piece print for the lower condyle jaw replica in an articulator. The printed portion is attachable and removable as discussed above allowing the permanent features of an articulator base, including the uprights as shown, to be re-used for other patients. FIG. 15 takes the device of FIG. 13B and shows attaching the printed lower jaw condyle structures (1502A, 1502B) to a more complete version of a lower base portion (1509) of the device. In other words, anatomically replicated condyles (1502A, 1502B) have been temporarily and removably attached to a horizontal connector (1509D) of a mechanical base portion (1509) for the articulator. As discussed above, the printed condyles (along with associated fossae) are printed by layered polymers such as stereolithographic three-dimensional printing techniques and methods.

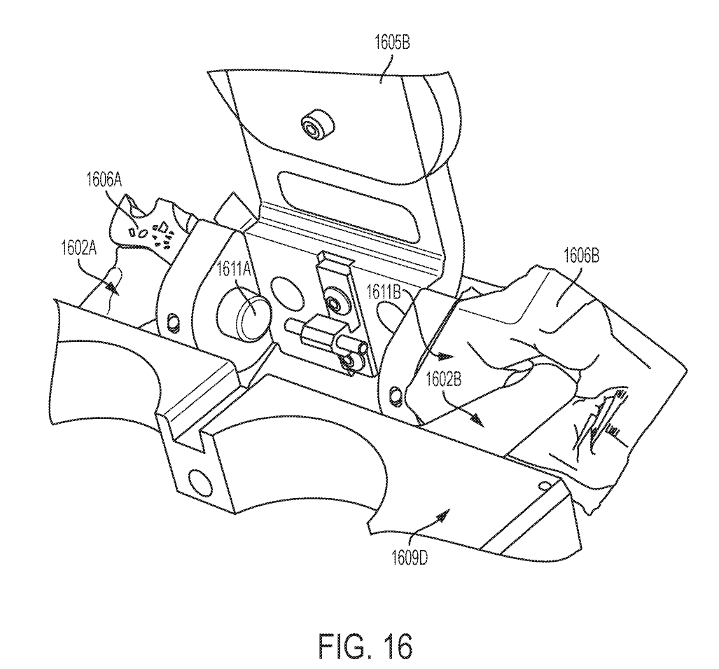

FIG. 16 illustrates the hinging effect that can be accomplished in an articulator according to this construction by utilizing a 3D print of the patient's mandibular fossae (1606A, 1606B) that receive the above referenced condyles as printed in the same format. FIG. 16 illustrates how the printed, or otherwise anatomically modeled, condyles (1602A, 1602B) and fossae for a patient can more accurately approximate an actual patient experience with a TMJ disorder and provide a much more accurate diagnostic tool for the physician and dentist to plan corrective action. Axles (1611A, 1611B) attach the printed or at least modeled fossae (1606A, 1606B) and attach the same to the upper frame (1605B) so that arcuate motion about the axles allow for adjusting fossae position. In the articulator of FIG. 15, the meeting of the condyles and fossae for the patient have the same boney projections, concave receptacles and convex condyle termini with irregularities that the mere spherical or angled replicas cannot match in accuracy. As can be discerned from an entire assembly, such as FIGS. 11 and 16, the base portion (1109, 1609) provides a stable reference point for mounting a modeled lower jaw on a platform that can be controlled for movement by the guide post (1113, 1613). An upper frame (1105B, 1605B) allows for rotational or arcuate motion about the horizontally oriented connector (1109D, 1609D) such that the upper frame may move, via fossae (1106A, 1106B, 1606A, 1606B), about anatomically modeled condyles (1102A, 1102B, 1602A, 1602B). The anatomically modeled condyles may be either statically attached to the articulator base in a vertical or upright position compared to a lower portion of the base (1109, 1609). With these assemblies, the upper frame (1105B, 1605B) mounts a model of a patient's upper jaw and moves that upper jaw with the upper frame within the confines of the manner in which the modeled condyles and modeled fossae of the real patient would move in the patient's body. In one embodiment, a handle extension (1809B) connected to the upper frame (1809) controls the rotational degree of freedom between the replica condyle and the replica mandibular fossa, and the control rod (1113) controls planar degrees of freedom in three dimensions for the mounting platform. An anatomical articulator according to this embodiment may include an axle connecting the mandibular fossa to the upper frame, wherein the rotational degree of freedom is exhibited by the upper frame about a longitudinal axis of the axle. An anatomical articulator therefore, may exhibit planar degrees of freedom in regard to a mounting platform of a lower base, i.e., the control rod (1113) may move the mounting platform in three dimensions about Cartesian x, y, and z axes.

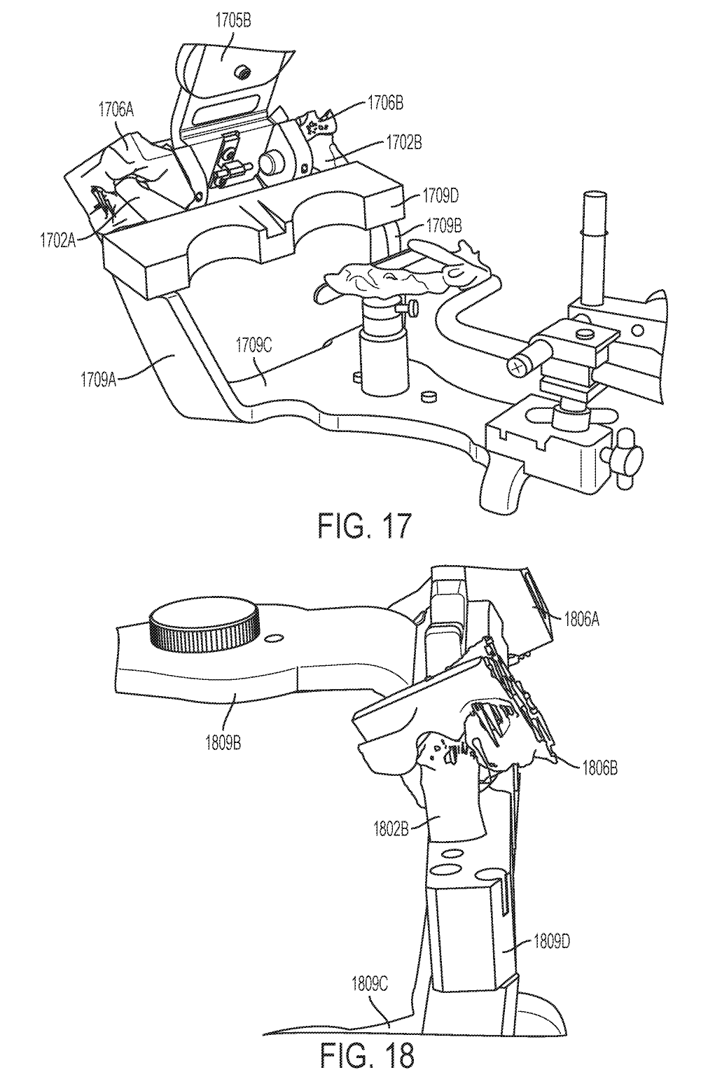

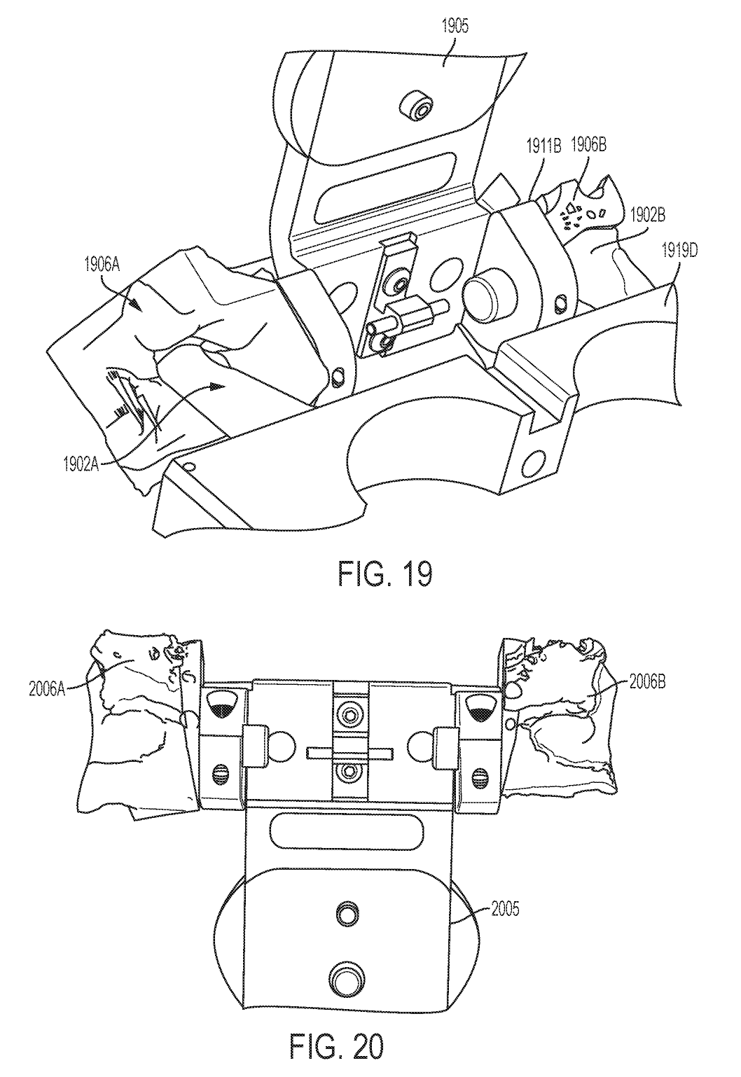

The closer images of FIGS. 17-20 emphasize the improvement in accuracy. In FIG. 17, the complex structure of condyles (1702) and the grooves and crevices of associated fossae (1706) are illustrated to emphasize the importance of modeling patient jaw movement accurately when dental molds are mounted on the upper frame and the lower base respectively. The side view of FIG. 18 illustrates an embodiment in which the upper frame includes an outward handle projection (1809B) for manipulating the upper frame, an associated mounted cast of an upper dental mold (not shown), and the fossae (1806A, 1806B) in relation to anatomically modeled condyles (1802A, 1802B). FIG. 19 illustrates the contours, crevices, and imperfections of the condyles (1902A, 1902B) as matched with the same kinds of rough hewn surfaces of the patient's fossae (1906A, 1906B). A bottom view as set forth in FIG. 20 is illustrative of the differences between anatomically modeled structures such as condyles and fossae (2006A, 2006N) and those used in prior art embodiments with perfect spheres and sharp angles for modeling the intersection in a temporomandibular joint.

The apparatuses described in this disclosure are the product of a diagnostic method by which a CBCT scan of a patient can be filtered, smoothed, and even digitally enhanced to provide a digital map for printing a patient's bone and teeth structures. The digital map can be used to make a 3D print of the patient's condyles and mandibular fossae for use as portions of a modular articulator as set forth herein. The printing techniques described herein are merely examples of how an anatomical model of a patient's TMJ can be achieved and other kinds of molds and casts utilizing polymeric materials, such as resins, are within the scope of this disclosure.

The software used for capturing a digital image of a patient's jaw structure may be compatible with many different image formats, whether the image is from magnetic resonance imagery (MRI) techniques or computer tomography techniques. Newer ways to digitize a shape of a patient's jaw structure cannot be ruled out as digital imaging with computer implemented processors, hardware, associated memory, and computer implemented software instructions are developed.

1. An anatomical articulator for modeling a temporomandibular joint comprising:
a lower base portion comprising at least one upright support;
a set of replica condyles produced from digital data representing a patient a structure, wherein the set of replica condyles connect to the at least one uptight support;
an upper frame portion defining receptacles on either side;
a set of replica mandibular fossae produced from the digital data representing a patient jaw structure and configured to connect to said upper frame portion via the receptacles;
wherein the replica condyles and the replica mandibular fossae meet to form a hinge of the articulator.

2. An anatomical articulator according to claim 1, wherein said set of replica condyles is removable from said upright support.

3. An anatomical articulator according to claim 1, wherein said replica mandibular fossae define axle portions that fit within the receptacles of the upper frame portion, allowing the upper frame portion to rotate about the replica condyles.

4. An anatomical articulator according to claim 1, wherein the hinge moves as allowed by the respective shapes of the replica condyles and the replica mandibular fossae.

5. An anatomical articulator according to claim 1, wherein the lower base portion and the upper frame portion are configured to each mount a dental cast.

6. An anatomical articulator according to claim 1, wherein the replica mandibular fossae are separable from the upper frame portion and from each other.

7. An anatomical articulator according to claim 1, wherein the digital data comprises digital data originating from a CBCT scan.

8. An anatomical articulator according to claim 1, wherein the replica condyles and the replica mandibular fossae comprise a resin material suitable for three-dimensional printing from the digital data.

9. An anatomical articulator for modeling motion of dental casts, comprising:
a lower base portion comprising at least one replica condyle attached to and extending from the lower base portion;
an upper frame portion supporting at least one replica mandibular fossa in a position to mate with the at least one replica condyle;
wherein the replica condyles and the replica mandibular fossae meet to form a hinge of the articulator, wherein the hinge exhibits a rotational degree of freedom between the at least one replica condyle and at least one replica mandibular fossa; and
wherein the rotational degree of freedom is limited by respective shapes of the replica condyle and the replica mandibular fossa.

10. An anatomical articulator according to claim 9, wherein the replica condyle and the replica fossa are removable from the respective lower base portion and the upper frame portion.

11. An anatomical articulator according to claim 10, wherein the upper frame portion and the lower base portion are reusable after removal of the replica condyle and the replica fossa.

12. An anatomical articulator according to claim 10, further comprising an axle connecting the mandibular fossa to the upper frame.

13. An anatomical articulator according to claim 10, further comprising a handle extension connected to the upper frame portion for controlling arcuate movement of the replica mandibular fossa about the replica condyle.

14. An anatomical articulator according to claim 13, further comprising a control rod connected to the lower base portion for controlling motion of a cast mounting platform connected to the lower base.

15. An anatomical articulator according to claim 14, wherein the handle extension controls the rotational degree of freedom between the replica condyle and the replica mandibular fossa, and the control rod controls planar degrees of freedom in three dimensions for the mounting platform.

16. An anatomical articulator according to claim 15, further comprising an axle connecting the mandibular fossa to the upper frame, wherein the rotational degree of freedom is exhibited by the upper frame about a longitudinal axis of the axle.

17. An anatomical articulator according to claim 15, wherein the planar degrees of freedom are exhibited about Cartesian x, y, and z axes.

18. A method of constructing an anatomical articulator, comprising:
digitally imaging a temporomandibular joint to form a digital model of the temporomandibular joint;
forming anatomical replicas of at least one condyle and at least one mandibular fossa, wherein the anatomical replicas represent portions of the temporomandibular joint that mate in the digital model;
attaching the replica mandibular fossa to an upper platform of the anatomical articulator;
attaching the replica condyle to a lower base of the anatomical articulator;
wherein the replica mandibular fossa and the replica condyle are configured to mate and connect the upper frame and the lower base.

19. A method according to claim 18, further comprising removing the replica mandibular fossa and the replica condyle from the articulator and re-using the lower base and the upper platform for different replica condyles and different replica mandibular fossae.

20. A method according to claim 18, further comprising attaching a dental cast corresponding to the digital model to at least one of the upper frame and the lower base.
